(19) United States
(12) Patent Application Publication
(10) Pub. No.: US 2005/0287096 A1
(43) Pub. Date: Dec. 29, 2005

(54) Method for improving the appearance of skin

(76) Inventor: Elias W. Packman, Merion Station, PA (US)

Correspondence Address: REED SMITH LLP, 2500 ONE LIBERTY PLACE, 1650 MARKET STREET, PHILADELPHIA, PA, 19103, US

(21) Appl. No.: 10/875,132

(22) Filed: Jun. 23, 2004

(51) Int. Cl. A61K 7/06; A61K 7/11

(52) U.S. Cl. 424/70.13

(57) ABSTRACT

A method for improving the appearance of skin is disclosed. The method includes applying a skin treatment technique to the human skin and applying to the skin a treatment composition wherein the treatment composition includes a disaccharide polysulfate-aluminum compound. Skin treatment techniques include chemical peels, skin whitening, removal of actinic keratosis, removal of basal cell carcinoma, mechanical peels, photo-activated peel and laser resurfacing.

### FIELD OF THE INVENTION

The present invention is directed generally to dermatological compositions based upon disaccharide polysulfate-aluminum compounds and methods for their use. More specifically, the invention relates to methods of using dermatological compositions based upon sucralfate with skin resurfacing techniques.

### DESCRIPTION OF THE BACKGROUND

The human body's largest organ is the skin and is composed of three layers: the epidermis, dermis, and subcutaneous fatty tissue. The skin varies in thickness from 0.5 mm on the eyelids to 4 mm or more on the palms and soles. The epidermis, the thinnest and outer most layer of skin, generally has a thickness of about 0.25 mm in the region of the face. The outer layer of the epidermis is sometimes known as the corneum layer and is generally composed of dead, hard, tough cells that form the hard surface of the skin. The epidermis is stratified into several layers, including the stratum basale, stratum spinosum, stratum granulosum, stratum lucidum and stratum corneum. The dermis which is typically 20-30 times thicker than the epidermis, contains hair follicles, sebaceous glands, nerve endings and fine blood capillaries. By volume the dermis is made up predominantly of the protein collagen. Subcutaneous fatty tissue is the deepest layer of the skin and is composed of connective tissue, blood vessels, and fat cells. This layer binds the skin to underlying structures, insulates the body from cold, and stores energy in the form of fat.

A common aim of many cosmetic procedures is to improve the appearance of a patient's skin. For example, a desirable clinical effect in the field of dermatology is to provide an improvement in the texture of aging skin and to give it a more youthful appearance. These effects can be achieved by the removal of a part or all of the epidermis, and on occasions part of the dermis, causing the growth of a new epidermis having the desired properties. Additionally, skin frequently contains scar tissue, the appearance of which is considered by some people to be detrimental to their attractiveness. The skin structure which gives rise to scar tissue is typically formed in the dermis. By removing the epidermis in a selected region and resculpting the scar tissue in the dermis it is sometimes possible to improve the appearance of certain types of scars, such as for example scars left by acne. The process of removing epidermal and possibly dermal tissue is known as skin resurfacing or dermabrasion.

There are many known techniques for achieving skin resurfacing, including, for example: the mechanical removal of tissue by means of an abrasive wheel; a technique known as a chemical peel which may involve the application of a corrosive chemical to the surface of the epidermis, to remove epidermal, and possibly dermal skin cells; the chemical bleaching of the skin, known as skin whitening; the removal through various techniques such as, for example, photo-dynamic therapy, for the removal of actinic keratosis and basal cell carcinomas; and laser resurfacing of the skin, whereby a controlled amount of energy is delivered to the epidermis causing necrosis of epidermal cells.

However, these skin treatment techniques often cause significant irritation to the patient, through the removal of the skin itself and latent side effects. For example, in addition to the irritation caused by removing the epidermal layer of skin, a mechanical or chemical peel may undesirably remove a portion of the dermal layer causing significant latent irritation to the patient while the dermal layer heals. Further, laser resurfacing, in addition to causing molecular dissociation within the cells of the epidermal layer, as a side effect raises the temperature of the skin which may lead to unwanted damage and irritation to the dermal layer.

Therefore, the need exists for a method for improving the appearance of skin that reduces the amount of irritation caused by the removal of layers of the skin.

### BRIEF SUMMARY OF THE INVENTION

The present invention is directed to a method for improving the appearance of human skin, comprising the steps of applying a skin treatment technique to the skin; and applying to the skin a treatment composition which includes a disaccharide polysulfate-aluminum compound.

Furthermore, the present invention includes a method of applying to the skin a chemical agent suitable for causing the skin to peel and applying to the skin a treatment composition including sucralfate.

The present invention solves problems experienced with the prior art because it provides a method for improving the appearance of skin that reduces the amount of irritation caused by the removal of layers of the skin. Those and other advantages and benefits of the present invention will become apparent from the detailed description of the invention hereinbelow.

### DETAILED DESCRIPTION OF THE INVENTION

It is to be understood that the descriptions of the present invention have been simplified to illustrate elements that are relevant for a clear understanding of the present invention, while eliminating, for purposes of clarity, many other elements found in typical dermatological applications. Those of ordinary skill in the art will recognize that other elements are desirable and/or required in order to implement the present invention. However, because such elements are well known in the art, and because they do not facilitate a better understanding of the present invention, a discussion of such elements is not provided herein.

In a particular embodiment of the invention, topical administration of a composition containing sucralfate is used as part of skin treatment techniques such as, for example, mechanical and chemical peels, skin bleaching, photo-dynamic therapy, and laser resurfacing, and the like.

Topical administration of a composition containing sucralfate applied post treatment will heal the damage to the skin caused by the stripping away of at least a portion of the epidermis layer. The compositions are administered once or several times a day for from one day to a week or more until the healing occurs.

The amount of the treatment composition of this invention that is to be applied to the skin will vary with a number of factors, including the severity of the skin treatment and the sensitivity of the affected skin area. Consequently, this will be a matter for the clinical judgement of the patient's health care professional. Generally, the treatment composition of the invention includes sucralfate in an amount from about 0.01% to 25% and may be administered in a range of from about 0.01 to about 2.0 grams per dose, not to exceed about 10 grams in a 24-hour period.

As observed in U.S. Pat. No. 4,945,084, which is incorporated in its entirety by reference herein, knee abrasions on basketball players, which were caused by falls during a basketball game, were treated with an ointment containing 5% sucralfate causing fusion to be stopped after twenty four hours with good healing occurring over a three day period thereafter.

Sucralfate may form a complex with tissue associated with wounded tissue, such as a layer of skin exposed due to forced peeling away of the top layer. Thus an embodiment of the present invention is the use of a composition containing sucralfate as a carrier composition for an additional pharmaceutical compound or compounds. The sucralfate may complex with the wounded area thereby holding the additional pharmaceutical compound near the wound. In this manner, for example, an antibiotic, a steroid, an antifungal agent, a biocidal agent, a local anesthetic or an anti-acne agent, or a combination thereof, is applied topically to a wound or lesion site in a composition containing sucralfate and is kept in place by the complexing action of the sucralfate with the tissues in the wound or lesion area.

For the topical treatment of wounds associated with treatments of the skin, the treatment composition may take the form of an ointment, cream, gel, paste, pad, liquid, foam or aerosol or any other composition suitable for topical administration. In other aspects, the composition of the invention may contain conventional materials and ingredients and conform to pharmacologically accepted formulations.

Treatment techniques of the skin may include mechanical alteration of the skin. As exemplified by U.S. Pat. No. 6,716,221, and incorporated in its entirety by reference herein, mechanical skin peeling devices often include a shaving apparatus in which a lower blade cooperating with an upper blade is driven to and fro parallel in a linear drive direction. This mechanical stripping to the human skin may cause significant and unwanted damage to the epidermis and, more specifically, to the dermis, given the generally rough nature of the skin surface and the impreciseness of a human controlled device. Such damage may result in severe irritation, scabbing and possible scarring of the skin. In an embodiment of the present invention, the treatment composition may be used in conjunction with the mechanical peeling of the skin to treat irritation and wounding of the skin.

Chemical peels use a chemical solution to improve and smooth the texture of the facial skin by removing its damaged outer layers. Chemical peels may also remove pre-cancerous skin growths, soften acne facial scars and even control acne. Chemical solutions suitable for chemical peels include: phenol, trichloroacetic acid (TCA), and alphahydroxy acids (AHAs), for example. AHAs, such as glycolic, lactic, or fruit acids are the mildest of the peel formulas and produce light peels. These types of peels may provide smoother, brighter-looking skin and require a short recovery period as compared to a phenol or TCA peel. TCA may be used in many concentrations, and may be most commonly used for medium-depth peeling. Fine surface wrinkles, superficial blemishes and pigment problems are commonly treated with TCA. The results of a TCA peel may be less dramatic nor as long-lasting as those of a phenol peel. Recovery time for a patient from a TCA peel is usually shorter than with a phenol peel. Phenol, the strongest of the chemical solutions, produces a deep peel and is generally used to treat patients with coarse facial wrinkles, areas of blotchy or damaged skin caused by sun exposure, or pre-cancerous growths.

AHA peels may cause temporary flaking or scaling, stinging, redness and dryness of the skin. Both a TCA peel and a phenol peel may cause significant swelling, depending on the strength of the peel used. Additionally, after a phenol or TCA peel, mild pain medication to relieve any tingling or throbbing may be necessary to help relief irritation and promote healing.

Treatment techniques of the skin may also include the treatment of pigmentation disorders. Pigmentation is the color of melanin, produced by pigment cells called melanocytes in the basal layer of the epidermis. It is produced by a chemical reaction, which involves an enzyme called tyrosinase. The melanin is then distributed to the other neighboring cells in the skin epidermis. Hyperpigmentation and hypopigmentation are two forms of skin disorders related to melanin production in the skin. Hyperpigmentation manifests itself in the form of dark patches from excess production of melanin, abnormal distribution of melanin in the skin, or possibly from the effects of drugs. Hypopigmentation manifests itself in the form of white patches on the skin caused by the loss or decreased production of melanin.

Melanin disorders, specifically hyperpigmentation, may be treated using skin bleaching techniques. This whitening of the skin may be accomplished by using active ingredients such as, for example, hydroquinone or kojic acid. Whitening agents are sold under several brand names, including, Claripel™ and Tri-Luma™, for example. Very often, the use of such whitening agents causes severe irritation, burning and dryness where topically applied on the skin. Specifically, labeling for the abovementioned brand name products each contain warnings related to skin irritation and hypersensitivity. In an embodiment of the present invention, the treatment composition may be used in conjunction with the use of whitening agents to treat and prevent unwanted side effects such as irritation, burning and dryness of the skin.

Other types of peels and skin treatment techniques may include the use of photo-activated agents, including, for example, 5-aminolevulinic acid and esters thereof. Examples of such compounds and methods may be found in U.S. Pat. Nos.: 6,710,066, 6,171,332 and 6,034,267, each incorporated by reference in their entirety. Such compounds are sold under various brand names, such as, for example, Metvix™. Photo-activated methods for treating the skin may include, by way of non-limiting example only, the treatment of non-malignant hyperproliferative skin lesions, actinic keratosis and basal cell carcinomas. Such techniques generally comprise administering to a patient an effective amount of a precursor of a photo-reactive compound, such as, for example, protoporphyrin IX, that may subsequently accumulate at therapeutic levels. Once a therapeutic level of a photo-activatable agent has been achieved, the treatment area is exposed to light capable of photo-activating the photo-activatable agent and may provide for the elimination of at least a portion of the lesion and skin. This disruption to the skin may create irritation to the patient and may require substantive healing of the treated area. In an embodiment of the present invention, the treatment composition may be used in conjunction with photo-dynamic therapy to treat and prevent unwanted side effects such as irritation, burning and dryness of the skin.

Yet other auxiliary ingredients may be employed in the composition of the present invention. In some instances, the composition may be formulated into a peel-off mask by employing a film forming carrier such as polyvinyl alcohol or an inorganic, mud pack type carrier such as a clay-based material. As is known in the art, colorings, fragrances, preservatives and the like may be similarly included in the compositions of the present invention.

The following examples are not intended to limit the invention but are merely illustrative thereof. It is understood that one of average skill in the art would be able to make substitutions, change proportions, make other variations, all within the scope of the teachings and without departing from the spirit of the invention and without undue experimentation.

### EXAMPLE 1

Performance of a Chemical Peel and Application of an Ointment.

A chemical peel using phenol may be performed on a patient.

An ointment may be formulated by mixing the following ingredients:

| Ingredient | % by Weight |
|---|---|
| Stearic Acid | 7 |
| Cetyl Alcohol | 2 |
| Mineral Oil | 20 |
| Glycerin | 10 |
| Triethanolamine | 2 |
| Purified Water qs | 100 |

To this ointment, there is added sucralfate to obtain a 5% final concentration. The ointment may be applied to a patient's skin post peel treatment.

### EXAMPLE 2

Performance of a Photo-Activated Peel and Application of an Gel.

5-Aminolevulinic acid may be introduced into a patient. Once a therapeutic level of the photo-activated agent is present, a light may be used to activate the peel.

A gel composition may be prepared by mixing the following ingredients:

| Ingredient | % by Weight |
|---|---|
| Samuet | 0.3 |
| Purified Water qs. | 100 |

To this gel there is added sucralfate to obtain a 5% final concentration.

The gel may be applied to a patient's skin post peel treatment.

### EXAMPLE 3

Performance of Laser Resurfacing and Application of an Cream.

A cream may be prepared by mixing the following ingredients:
5% Sucralfate
qs. cream

The cream is preferably applied to the patient's skin as the final step of the laser resurfacing treatment.

### EXAMPLE 4

Performance of Skin Whitening and Application of an Ointment

An ointment may be prepared by mixing the following ingredients:

| Ingredient | % by Weight |
|---|---|
| Preparation H | 95 |
| Sucralfate | 5 |

The ointment may be applied to a patient's skin after the skin whitening treatment has been performed.

### EXAMPLE 5

Performance of a Skin Treatment Technique and Application of an Ointment.

An ointment was formulated by mixing the following ingredients:

| Ingredient | % by Weight |
|---|---|
| Stearic Acid | 7 |
| Cetyl Alcohol | 2 |
| Mineral Oil | 20 |
| Glycerin | 10 |
| Triethanol Amine | 2 |
| Benzocaine | 6 |
| Sucralfate | 5 |
| Purified Water | 48 |

The ointment may be applied to a patient's skin before and after the Skin Treatment Technique has been performed.

The disclosure herein is directed to the variations and modifications of the elements and methods of the invention disclosed that will be apparent to those skilled in the art in light of the disclosure herein. Thus, it is intended that the present invention covers the modifications and variations of this invention, provided those modifications and variations come within the scope of the appended claims and the equivalents thereof.

1. A method for improving the appearance of skin, comprising the steps of:
applying a skin treatment technique to the human skin; and
applying to said skin a treatment composition wherein said treatment composition includes a disaccharide polysulfate-aluminum compound.

2. The method of claim 1, where said disaccharide polysulfate-aluminum compound is sucralfate.

3. The method of claim 1, wherein said skin treatment technique includes one selected from the group consisting of a chemical peel, skin whitening, removal of actinic keratosis, removal of basal cell carcinoma, removal of squamous cell carcinoma, a mechanical peel, photo-activated peel and laser resurfacing.

4. The method of claim 1, wherein said chemical peel is selected form the group consisting of phenols, trichloroacetic acid, and alphahydroxy acids.
